(19) United States
(12) Patent Application Publication
(10) Pub. No.: US 2022/0409483 A1
(43) Pub. Date: Dec. 29, 2022

(54) Connector and Connection System for a Medical Packaging, and Method for Providing a Liquid for a Medical Packaging

(71) Applicant: Fresenius Kabi Deutschland GmbH, Bad Homburg (DE)

(72) Inventors: Torsten Brandenburger, Friedberg (DE); Janine Seidel, Friedberg (DE); Christian Nitsche, Friedberg (DE)

(21) Appl. No.: 17/777,823

(22) PCT Filed: Dec. 10, 2020
(86) PCT No.: PCT/EP2020/085437

§ 371 Date: May 18, 2022

(30) Foreign Application Priority Data
Dec. 17, 2019 (DE) 19216861.5

(51) Int. Cl. A61J 1/14; A61J 1/10

(52) U.S. Cl. CPC A61J 1/1418; A61J 1/10

(57) ABSTRACT

The invention relates to a connector for a medical packaging. The connector comprises an attachment part with a sealing element, the sealing element having a sealing head with at least one slit. The sealing head sits in an inner sleeve of the attachment part, the sealing head being adjoined by at least one trunk portion, which has a larger outer diameter than the sealing head. A gap is present between the at least one trunk portion and an opposite inner wall of the attachment part. An interior of the sealing element is free from a body with a tip that is intended to assist the opening of the sealing head. The attachment part has an outer sleeve which surrounds the inner sleeve and which has an internal thread.

The invention relates to a connector for a medical packaging, a medical packaging equipped with the connector as well as a connection system, which comprises the connector.

The invention also relates to an apheresis set as well as a method for providing a fluid connection to a medical packaging.

### BACKGROUND OF THE INVENTION

Connection systems for medical packagings, in particular for infusion bags, are known in a plurality of variants in practice.

Such connection systems generally comprise a septum, which seals the connector and which is pierced with a spike or with a needle to establish a fluid connection.

Such a connection system for medical containers is for example shown in the document WO 2016/188957 A1 (Applicant: Fresenius Kabi Deutschland GmbH).

Connection systems according to the so-called Correct Connect Standard are used in particular for apheresis sets. This standard is intended to provide a standardized connection system, in which the hose connection comprises an external thread via which the hose connection is connected to the connector.

The connection system known from the published document mentioned above is therefore not suitable for the Correct Connect Standard.

A male connector is described in the document US 2019/0184152 A1 for connecting two hose lines. A valve body is movably arranged in the connector. A hollow-shaped body is provided with a tip in the interior of the valve body, said tip assisting the opening of the valve body. In the connected state of the connector, the hollow-shaped body pierces the sealing head of the valve body with its tip.

It has been shown that it is difficult to provide a connector for the Correct Connect System in which the connector is reliably sealed after the connection has been unscrewed.

### OBJECT OF THE INVENTION

Against this background, the object underlying the invention is to provide a connector, which has an internal thread and which ensures sealing, with a high level of reliability, when the connection piece is unscrewed. In particular, in the connected state, the flow should also essentially not be impaired. Additionally, it should be possible to provide the interior of the connector in particular so as to be sterile.

### SUMMARY OF THE INVENTION

The object of the invention is already achieved by a connector or a connection system with a connector as well as by a method for providing a fluid connection to a medical packaging according to one of the independent claims.

Preferred embodiments and further developments of the invention can be inferred from the subject matter of the dependent claims, the description as well as the drawings.

The invention relates to a connector for a medical packaging, with the connector comprising an attachment part with a sealing element and the sealing element having a sealing head with at least one slit. The sealing head sits in an inner sleeve of the attachment part, with the sealing head being adjoined by at least one trunk portion, which has a larger outer diameter than the sealing head. A gap is present between the at least one trunk portion and an opposite inner wall of the attachment part. The attachment part has an outer sleeve which surrounds the inner sleeve and which has an internal thread. In particular, an interior of the sealing element is free from a body, in particular a hollow body, with a tip, which is intended to assist the opening of the sealing element, in particular of the sealing head. This will not essentially impair the flow in the connector.

It has been found that a connector designed in this way and interacting with a connection can form a system in which the connection comprising a connection inner sleeve for providing a fluid connection can be screwed in and unscrewed again, with sealing of the connector being reliably ensured after the connection has been removed.

The sealing element comprises a sealing head with at least one slit. If the sealing head expands in its diameter, an opening will be released through the slit which provides a fluid connection and into which the connection inner sleeve can penetrate.

A trunk portion, which adjoins the sealing head and which has a larger outer diameter than the sealing head and which is surrounded by a gap, means that when the connection is attached and the connection head pushed back through the connection inner sleeve, the trunk portion can expand and can therefore also expand the sealing head in a defined manner.

The connector preferably comprises a lower part with an upper part designed as an attachment part.

The attachment part is connected to the lower part, in particular snapped onto the lower part.

Through the attachment part, an inner sleeve is provided into which the connection inner sleeve can be pushed.

The inner sleeve is surrounded by an outer sleeve with an internal thread, which serves to screw in the connection outer sleeve.

The connector is thus designed at least in a front region with two walls and comprises the inner sleeve, which is surrounded at least in sections by the outer sleeve with the internal thread.

The sealing head preferably protrudes into the inner sleeve in the non-connected state.

However, since the thread extends only over a front region of the connector, the attachment part, viewed from the front, merges into a single-walled configuration behind the thread of the outer sleeve.

This transition is preferably in a central region of the sealing element in the non-connected state.

The sealing element consists of an elastic material, in particular of a polyurethane or a silicone or comprises these materials.

According to one embodiment of the invention, a front surface of the sealing head is spaced apart from a front-side end of inner sleeve.

The inner sleeve thus protrudes beyond the outer sleeve, with the sealing head of the sealing element preferably being spaced apart from the front-side end of the inner sleeve.

This means that the connection can be easily inserted into the inner sleeve and that the thread of the connection already engages before a resistance is produced by sealing element by attaching the tip of the connection inner sleeve.

Thus, an easy and secure attachment of the connection is ensured.

In the case of one embodiment of the invention, the sealing element has a flange, which is arranged between the attachment part, which is designed as an upper part, and a lower part.

A defined positioning can be achieved by the flange, which is preferably attached on a rear region of the sealing element.

In the case of one embodiment of the invention, the trunk portion comprises at least one shoulder, which abuts on a step of the attachment piece.

It is in particular provided that the sealing element has two steps, with a front step inside the region of the sealing head and a rear step forming the transition to the trunk portion within the meaning of the invention.

At least one step, in particular the step at which the sealing head merges into the trunk portion, at the same time forms the transition behind which a gap extends around the sealing element.

In the non-connected state, the head piece of the sealing element thus abuts on the inner wall of the attachment part, whereas the trunk portion is spaced apart by a gap.

When being connected, the trunk portion can thus expand and spread radially into the gap.

The transition from the sealing head to the trunk portion is preferably at the height of a transition region in which the attachment part merges from a two-walled configuration to a single-wall configuration.

Thus, the gap can be arranged radially behind the thread in place of the thread. A particularly compact configuration of the connector is thus ensured.

In the case of one embodiment of the invention, the sealing head comprises a front portion, which merges via a step into a rear portion with a larger outer diameter, with the step of the sealing head abutting on a step of the inner sleeve.

Thus, a defined expansion of the sealing head is ensured when the connection inner sleeve is introduced.

The sealing head is pushed back when the connection is screwed in, with the front portion of the sealing head being merged into the region in which the rear portion of the sealing head was previously arranged in the non-connected state.

The front portion is thus expanded roughly by the difference of the diameter from the front portion to the rear portion and releases the channel into which the connection inner sleeve initially abutting on the front side of the sealing head can penetrate.

The step between the front and the rear portion of the sealing head and/or the step of the inner sleeve is preferably rounded or designed running obliquely, in particular in a ramp shape, to enable a slight sliding back and forth.

The attachment part can be sealed with a break-off part. As a result, the interior of the connector can for example be provided so as to be sterile. Additionally, such a break-off part represents a tamper-evident closure.

The break-off part preferably has at most the diameter of the inner sleeve.

The break-off part is thus preferably attached only on the inner sleeve.

In particular, the inner sleeve can be designed in one piece with the break-off part by, for example, a predetermined break point being designed on the front side of the inner sleeve by way of a constricted portion.

The break-off part can thus be designed particularly compactly and in a manner that saves materials.

The invention also relates to a medical packaging, which is in particular designed as a film bag or bottle of plastic or glass and which comprises a connector described above.

The invention also relates to a connection system with the connector described above as well as a connection, which has a connection inner sleeve designed to engage into the inner sleeve of the connector and which has a connection outer sleeve with an external thread.

The connection system is in particular designed in such manner that the connection inner sleeve pushes the sealing head back when the connection is screwed into the connector, with the trunk portion of the sealing element expanding in a hollow space formed by the gap and with the diameter of the sealing head enlarging such that the slit is opened and the channel is opened for the passage of the fluid.

In particular, the connection inner sleeve, which is preferably designed conically, penetrates into the channel and is thus sealed radially by the sealing head in the connected state.

In particular, the connection system is designed in such manner that, when the connection is screwed in, the front portion of the sealing head is pushed back into a region of the attachment part, in which the inner diameter of the attachment part, in particular behind a step, is enlarged.

Through the invention, a connection system is provided, which, in spite of resealability, is designed in such manner that the cross-section of the channel formed by the slit corresponds at least to the smallest inner cross-section of the connection inner sleeve.

The minimum cross-section of the fluid connection is thus not reduced by the connector in the connected state.

When the connection is unscrewed, the sealing set is in turn sealed by returning to its original shape.

The sealing head is designed in a preferred embodiment in such manner that the slit in the non-connected state has a length that is smaller than the outer diameter of the connection inner sleeve.

This ensures that the connection inner sleeve first attaches with its front side on the sealing head, then pushes the sealing head back and only when the sealing head has expanded does it penetrate into it.

The invention relates in particular to an apheresis set, which comprises the connection system described above.

The apheresis set thus comprises at least one hose connection to a medical packaging, in particular to a film bag, which is provided with the connection system according to the invention.

An anti-coagulant solution can in particular be provided via a medical packaging of the apheresis set.

The invention also relates to a method for providing a fluid connection to a medical packaging. The method is in particular carried out by means of the connection system described above and comprises the following steps:
providing a connector, which comprises an attachment part with a sealing element, with the sealing element having a front surface with at least one slit, and with the attachment part having an outer sleeve which surrounds an inner sleeve and which has an internal thread,
providing a connection, which has a connection inner sleeve designed to engage into the inner sleeve of the connector, and a connection outer sleeve with an external thread,
screwing the connection into the connector, with the connection inner sleeve pushing the front surface of the sealing element back, with the sealing element, in particular a sealing head of the sealing element, being expanded and enlarging in diameter such that the slit opens into a channel. In particular, an interior of the sealing element is free from a body, in particular a hollow body, with a tip, which is intended to assist the opening of the sealing element, in particular of the sealing head.

The opening channel is in particular designed in such manner that the connection inner sleeve penetrates into the channel when opened and is thus sealed radially.

For the method, the sealing element described above is in particular used, which has a sealing head with a front portion, which comprises the front surface with the slit, with the front portion being adjoined by a rear portion which has a larger diameter than the front portion, with the front portion being enlarged in its diameter behind a step of the attachment part when the sealing head is pushed back.

If the connection is unscrewed, the sealing element returns to its original shape, with the fluid connection being closed by the diameter of the sealing element, in particular of the sealing head, being reduced, while the front surface of the connection inner sleeve still sits on the sealing element in a sealing manner.

The sealing element thus contracts when the connection inner sleeve is moved back, with the tip of the connection inner sleeve still abutting ensuring at this moment that no fluid or only a minimum amount of fluid can leak out while returning to its original shape.

### BRIEF DESCRIPTION OF THE DRAWINGS

The subject matter of the invention will be explained in more detail below with reference to the drawings FIG. 1 to FIG. 13.

FIG. 1 is a perspective view of a medical packaging with a connector according to the invention.

In the corresponding representation according to FIG. 2, the break-off part of the connector is removed.

In the corresponding representation according to FIG. 3, the connection is represented, which will now be connected to the connector.

FIG. 4 shows the lower part of the connector in a perspective view.

FIG. 5 shows the sealing element in a perspective view.

FIG. 6 shows the upper part or attachment part in a perspective view.

FIG. 7 is a perspective view sectioned axially along the main extension direction of the connector, with the connector still not being connected to a connection, but the break-off part already being removed.

FIG. 8 is a corresponding sectioned perspective view of the connection.

FIG. 9 is an axial sectional view of the connector in the non-connected state.

FIG. 10 is a sectional view of the connector, with it being connected to a connection. The configuration of the sealing element in this representation schematically.

FIG. 11 is a detailed view of the region A of FIG. 10.

FIG. 12 is a photograph of the channel taken by means of a camera endoscope which is provided by the connection piece according to the invention and the connector.

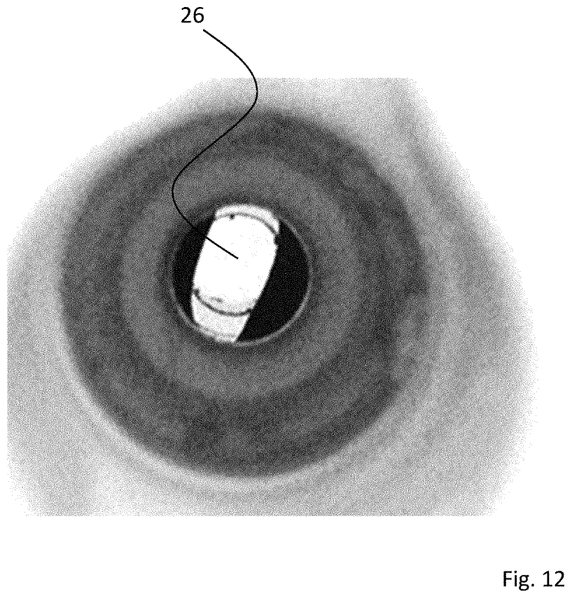

FIG. 13 shows the method steps according to an exemplary embodiment of the method according to the invention for providing a fluid connection.

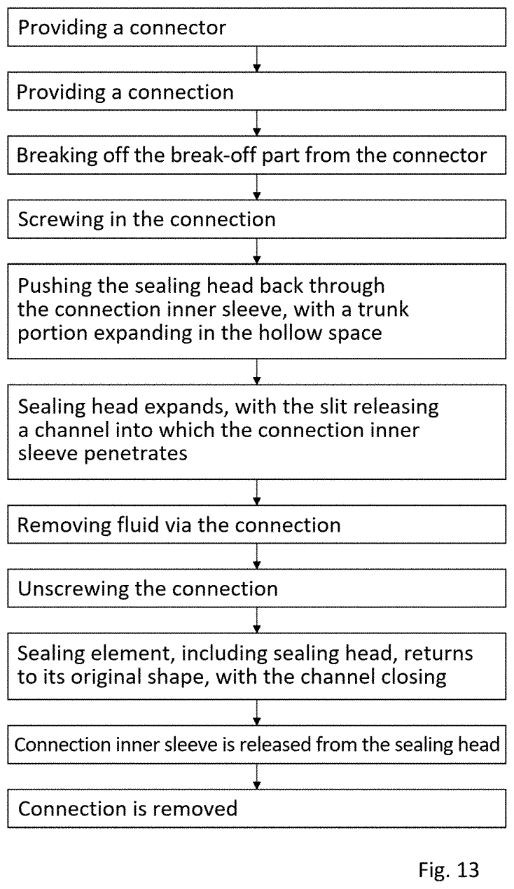

### DETAILED DESCRIPTION OF THE DRAWINGS

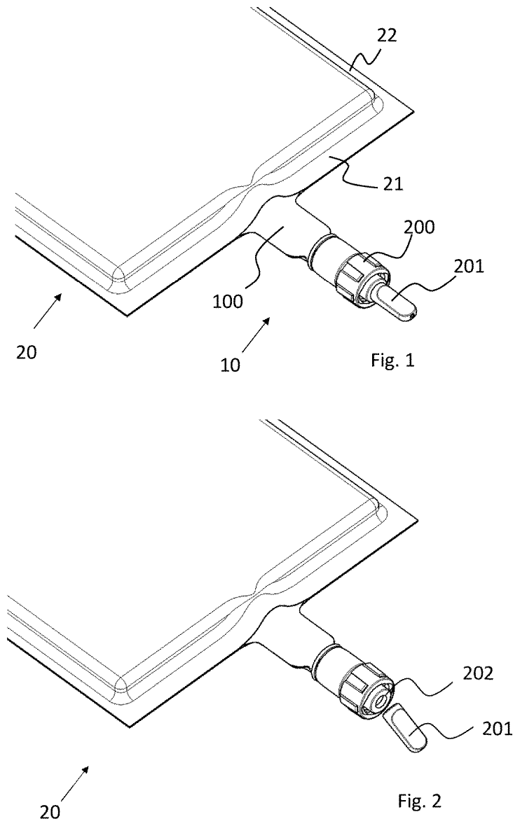

The use of a connection system according to the invention will be explained briefly with reference to FIG. 1 to FIG. 3.

FIG. 1 shows, in a perspective view, a medical packaging 20, which is connected to a connector 10 according to the invention.

The medical packaging 20 is designed as a film bag with a longitudinal welding seam 22 and a cross welding seam 21.

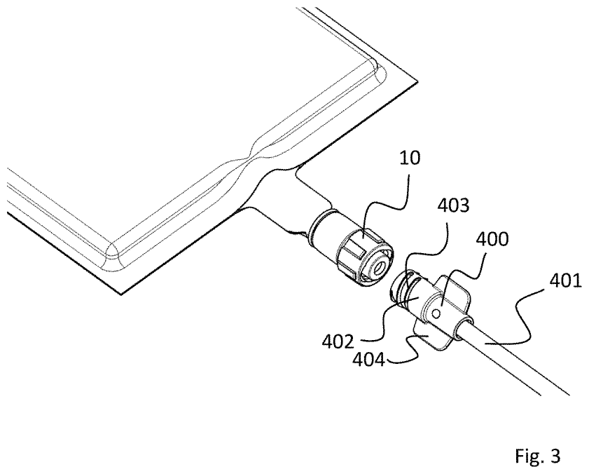

The connector 10 comprises a lower part with a weld-in portion 101 (see FIG. 4), which is welded in the cross welding seam 21 and therefore connected in a fluid-tight manner to the medical packaging 20 designed as a film bag.

The connector further comprises an upper part 200, which is designed as attachment part 200 attached on the lower part 100 and which has a break-off part 201.

As represented in FIG. 2, an inner sleeve 202 is released by removing the break-off part 201.

FIG. 3 is a perspective view in which the connection 400 of the connection system designed in a complementary manner is now represented.

The connection 400 is connected to a hose 401.

The medical packaging 20 and the connection system can thus be for example part of an apheresis set.

The connection 400 comprises a connection outer sleeve 402 designed to be screwed into the connector 10.

The connection outer sleeve 402 is therefore provided with an external thread 403.

In order to facilitate screwing-in, the connection 400 comprises handling aids which are designed in this exemplary embodiment as wings 404 extending radially outwards.

When the connection 400 is screwed in, a connection inner sleeve (405, cannot be seen in this view) is pushed into the inner sleeve 202 of the connector 10 and the connection 400 is screwed with the connector 10.

As will be represented in even more detail below, the penetrating connection inner sleeve 405 opens a sealing element 300 of the connector 10.

FIG. 4 is a perspective view of the lower part 100 of the connector 10.

The lower part 100 comprises, in this exemplary embodiment, a weld-in portion 101 for connecting to a medical packaging 20. In this exemplary embodiment, the weld-in portion 101 is ship-shaped designed , whereby it can be optimally welded in the welding seam 21 of a film bag. The invention, however, also relates to connectors attached in different manners.

The lower part 100 comprises a head 102, on which the upper part 200 designed as attachment part 200 is attached, in particular snapped in.

The head 102 comprises a collar 103, which can serve to snap in the upper part 200.

Axially running webs 104 are arranged above and below the collar 103 which act in cooperation with the upper part 200 as a torsion-proof connection.

The lower part 100 provides a channel 106, which is surrounded by an axially extending web 107.

An annular holder 105 for the sealing element 300 extends around the web 107.

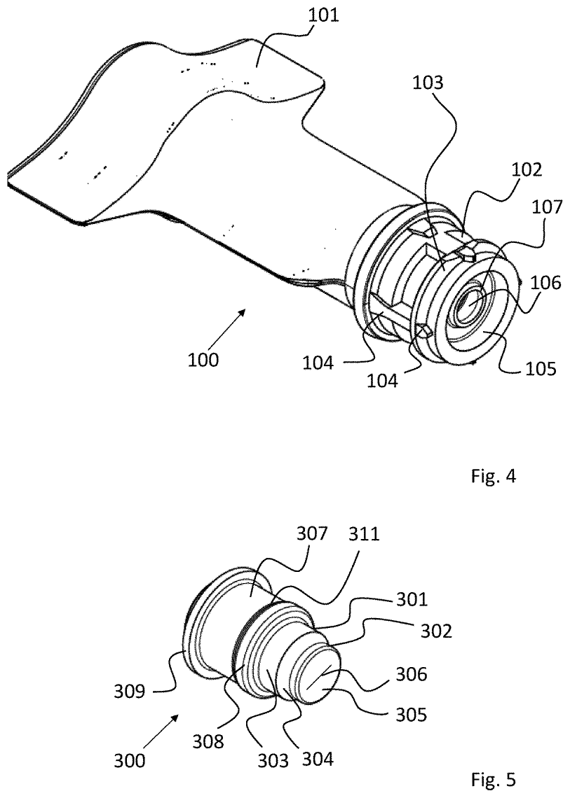

The sealing element 300 represented in FIG. 5 in perspective view is inserted into the holder 105 in the mounted state with the end portion 310.

Otherwise, the sealing element 300 consists of a sealing head 301 and a trunk portion 307. When viewed from front to back, the sealing head 301 first comprises a front portion 302, which comprises the front surface 305, which is provided with at least one slit 306 in order to form a fluid connection.

The front portion 302 merges via the step 304 into a rear portion 303 of the sealing head 301.

The step 304 is, in this exemplary embodiment, designed obliquely, i.e. in the form of a ramp, in order to enable a slight sliding back and forth of the sealing head 301.

The trunk portion 307 adjoins the rear portion 303 of the sealing head 301 via the shoulder 308, which also forms a step.

The trunk portion 307 in turn has a larger diameter than the rear portion 303 of the sealing head 301.

The shoulder 308 is located only in a front region of the trunk portion 307 such that the trunk portion merges via a step 311, also designed as a slope, into a portion, which has a smaller outer diameter. The inner diameter of the trunk portion 307 is, in contrast, constant.

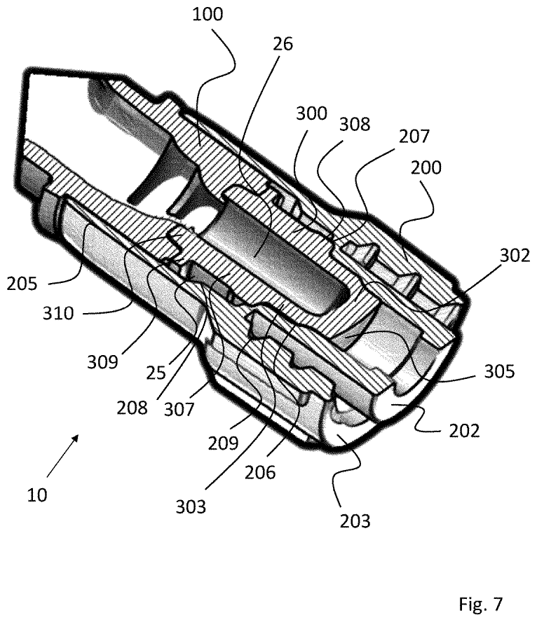

The flange 309 is arranged at the end of the trunk portion 307. Behind the flange 309, an end portion 310 (see FIG. 7) can also adjoin, which is designed as an axially protruding ring and which engages into the holder 105 of the lower part 100 in the connected state.

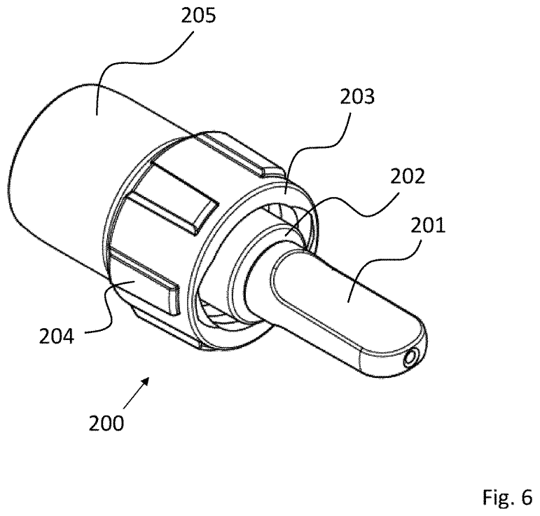

FIG. 6 shows, in a perspective view, the upper part 200 designed as attachment part 200.

The upper part 200 comprises a break-off part 201, which sits on the inner sleeve 202.

On the front side of the inner sleeve 202, at the transition to the break-off part 201 is arranged a circumferential weakening line, for example a constricted portion, which serves as the predetermined break point in order to break off the break-off part 201.

An outer sleeve 203, which has an internal thread 209 (see FIG. 7), extends around the inner sleeve 202.

This internal thread 209 serves to screw in the connection 400.

The outer sleeve 203 comprises an outer profiling 204, via which the connector 10 can be easily retained when the connection 400 is screwed in.

The rear end of the upper part 200 forms the snap-in portion 205, which is designed to snap on the head 102 of the lower part 100.

FIG. 7 shows, in an axially sectioned perspective view, the connector 10 with break-off part 201 already removed in the non-connected state.

The front side of the inner sleeve 202 protrudes beyond the front side of the outer sleeve 203.

A channel extends up to the front surface 305 of the sealing element 300 through the inner sleeve 202.

Proceeding from the medical packaging 20, a channel 26, which preferably has a substantially constant cross-section inside of the sealing element 300, runs up to the front-side wall of the front portion 302 of the sealing element 300.

The sealing element 300 thus seals the channel 26 in the non-connected state.

The slit 306 of the sealing element 300 represented in FIG. 5 is sealed.

The outer sleeve 203 comprises the internal thread 209, which is opposite the inner sleeve 202.

A front step 206 is located on the inner sleeve 202 in the region of the sealing head 301, which extends up to the height of the internal thread 209 in the non-connected state. The step 304 of the sealing element 300 abuts on the front step 206 in the non-connected state and forms the transition from the front portion 302 to the rear portion 303 of the sealing head 301.

Then, the upper part comprises a middle step 207, which is arranged directly behind the internal thread 209.

The shoulder 308 of the sealing element 300 abuts on the middle step 207 and forms the transition from the sealing head 301 to the trunk portion 307.

At the beginning of the middle step 207, the outer wall of the sealing element 300 no longer abuts on the inner wall of the upper part 200, but rather is spaced apart by a gap 25.

A hollow space is formed by the gap 25 into which hollow space the trunk portion 307 can expand when the sealing head 301 is pushed back through the connection inner sleeve 405.

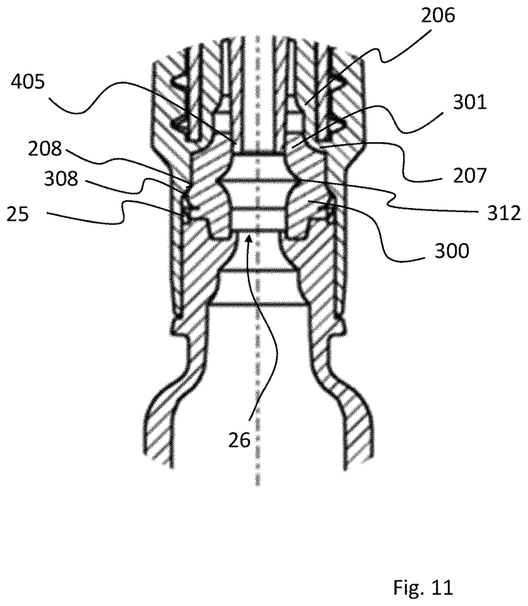

Additionally, the upper part 200 has, inside the trunk region, a rear step 208, which serves for defined support of the shoulder 308 of the trunk portion 307 in the connected state (see FIG. 11).

The position of the sealing element 300 is secured behind, i.e. on the side opposite the sealing head 301, by the end portion 310, which abuts on the inner wall of the holder 105 of the lower part 100.

The rear portion 310 of the sealing element 300 is secured axially by the flange 309, which sits on the front surface of the lower part 100.

The upper part 200 is connected in a form-fitting manner to the lower part 100 via the snap-in portion 205.

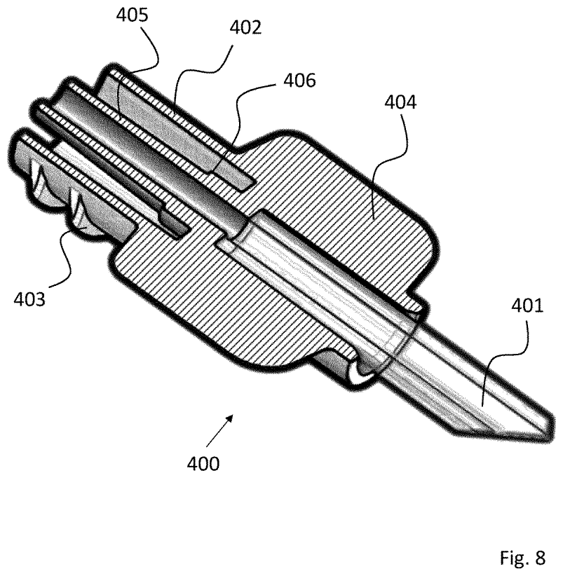

FIG. 8 also shows, in an axially sectioned perspective view, the connection 400, which, in this exemplary embodiment, comprises a hose 401, which can lead for example to an apheresis device.

The hose 401 is, in this exemplary embodiment, connected to the connection 400 with wings 404 inside the region.

At the front, the connection 400 comprises a preferably conically designed connection inner sleeve 405, which is surrounded by the connection outer sleeve 402 with the external thread 403.

When inserted into the connector 10, the connection inner sleeve 405 is first pushed into the inner sleeve 202 of the connector 10.

The connection 400 is thus already centered and can be easily pushed forwards until the external thread 403 of the connection 400 engages with the internal thread 209 of the connector 10. Pushing the connection 400 further forwards is then effected by screwing-in. By converting the rotational movement into an axial movement, larger forces do not thereby have to be applied by the user, although the sealing element 300 must now be deformed to provide a fluid connection.

The connection inner sleeve 405 can end with a step 406 or a stop, via which a defined position is ensured in the axial direction in the connected state.

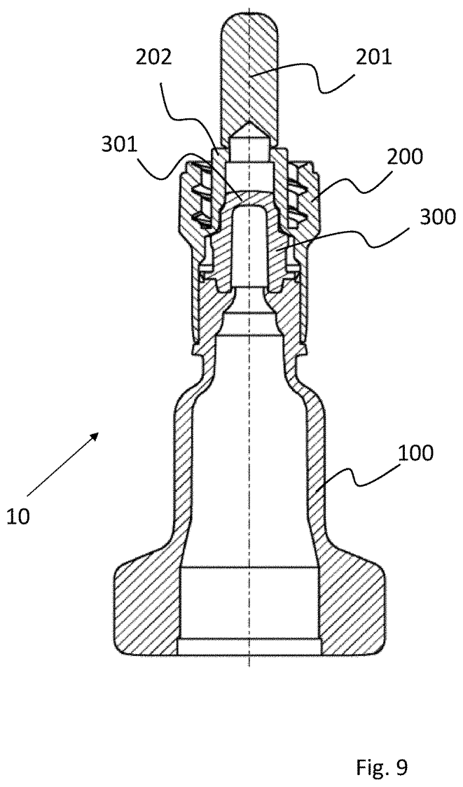

FIG. 9 shows, in an axial sectional view, the connector 10 in the non-connected state.

The break-off part 201, which seals the inner sleeve 202 and which has, in this exemplary embodiment, a smaller maximum diameter than the inner sleeve 202, is still attached.

The sealing element 300 is inserted between lower part 100 and upper part 200 such that the connector 10 is sealed by the sealing head 301.

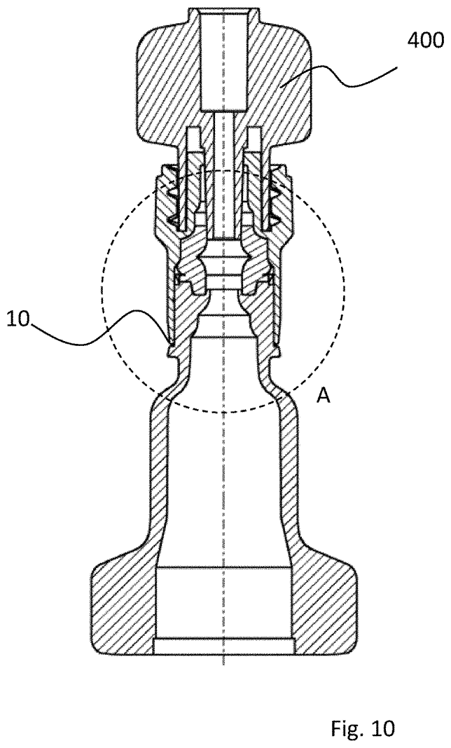

FIG. 10 shows, in an axial sectional view, how the connection 400 is now connected to the connector 10.

FIG. 11 is a detailed view of the region A of FIG. 10.

It can be discerned that the channel 26 now extends through the connector 10, consequently a fluid connection is provided.

The sealing head 301 has been pushed back from the front step 206 proceeding from the connection inner sleeve 202.

The front portion 302 of the sealing head 301 now sits behind the front step such that the front portion 302 has expanded and through the slit 306 a channel 26 has been formed into which the tip of the connection inner sleeve 405 has penetrated.

The shoulder 308 of the transition region to the trunk portion 307 now abuts in a defined position on the rear step 208 of the upper part 200.

Otherwise, the trunk portion 307 has been pushed into the gap 25. An axial movement of the sealing head 301 is possible through this material displacement into the gap 25.

The trunk portion 307 thereby forms a bulge or fold 312 on the inside.

When the connection 400 is unscrewed again, the sealing element 300 returns to its original shape and the connector 10 is sealed again.

The sealing element 300 does not for example represent a bellows, which can be axially compressed. The trunk portion 307 is substantially cylindrical. The wall of the trunk portion 307 is designed substantially flat.

FIG. 9 is a photograph of the channel 26 in the connected state taken by means of a camera endoscope, which is looking into the connection inner sleeve 405.

It can be discerned that the slit 306 has expanded in such manner that the connection inner sleeve 405 could penetrate into the sealing head 301 such that the minimum cross-section of the connection inner sleeve 405 is not reduced again by the connector 10.

FIG. 11 shows the individual steps of the method according to the invention for providing a fluid connection according to an exemplary embodiment of the invention.

First, a connector 10 and a connection 400 are provided.

Then, the break-off part 201 is separated from the connector 10.

The connection 400 is then introduced and screwed into the connector 10.

When being screwed in, the sealing head 301 is pushed back through the connection inner sleeve 405, with the trunk portion 307 expanding in the hollow space formed by the gap 25.

The sealing head 301 also expands, in particular so that a front portion 302 of the sealing head 301 is pushed behind a front step 206 of the attachment part 200.

This releases a channel 26 into which the connection inner sleeve 405 penetrates.

Then, fluid can be removed or supplied via the connection 400.

When the connection 400 is unscrewed, the sealing element 300, including the sealing head 301, returns to its original shape, with the channel 26 being closed.

In the moment when the front portion 302 of the sealing head 301 contracts again, the connection inner sleeve 405 still sits on the front surface 305 of the sealing head 301, whereby fluid is prevented from leaking out.

Then, the connection inner sleeve 405 is released from the sealing head 301 and the connection 400 can be removed again.

Through the invention, a connector 10 can be provided which is suitable in particular for the Connect Correct System, which is safe to handle and is resealed when the connection 400 is removed.

### LIST OF REFERENCE NUMERALS

10 Connector

20 Medical packaging

21 Cross welding seam

22 Longitudinal welding seam

25 Gap/hollow space

26 Channel

100 Lower part

101 Weld-in portion

102 Head

103 Collar

104 Web

105 Holder for sealing element

106 Channel

107 Web

200 Attachment part/upper part

201 Break-off part

202 Inner sleeve

203 Outer sleeve

204 Profiling

205 Snap-in portion

206 Front step

207 Middle step

208 Rear step

209 Inner thread

300 Sealing element

301 Sealing head

302 Front portion

303 Rear portion

304 Step between front and rear portion

305 Front surface

306 Slit

307 Trunk portion

308 Shoulder

309 Flange

310 End portion

311 Step

312 Fold

400 Connection

401 Hose

402 Connection outer sleeve

403 Outer thread

404 Wing

405 Connection inner sleeve

406 Step/stop

1. A connector for a medical packaging, comprising an attachment part with a sealing element, wherein the sealing element has a sealing head with at least one slit, wherein the sealing head sits in an inner sleeve of the attachment part, wherein the sealing head (301) is adjoined by at least one trunk portion, which has a larger outer diameter than the sealing head, wherein an interior of the sealing element is free from a body with a tip, which is intended to assist the opening of the sealing element and wherein a gap is present between the at least one trunk portion and an opposite inner wall of the attachment part, and wherein the attachment part has an outer sleeve, which surrounds the inner sleeve and has an internal thread and wherein the attachment part is sealed with a break-off part.

2. The connector according to the preceding claim 1, characterized in that a front surface of the sealing head is spaced apart from a front-side end of the inner sleeve.

3. The connector according to claim 1, characterized in that the connector is designed to be resealable.

4. The connector according to claim 1, characterized in that the sealing element has a flange, which is clamped between the attachment part, which is designed as an upper part, and a lower part.

5. The connector according to claim 4, characterized in that the trunk portion has a shoulder, which abuts on a step of the attachment piece, and/or in that the sealing head has a front portion, which merges via a step into a rear portion with a larger outer diameter, wherein the step abuts on a step of the inner sleeve.

6. The connector according to claim 5, characterized in that the step is rounded between the front portion and the rear portion of the sealing head and/or the step of the inner sleeve is rounded.

7. The connector according claim 1, characterized in that the break-off part sits on the inner sleeve (202).

8. The connector according to claim 1, characterized in that the inner sleeve protrudes beyond the outer sleeve.

9. A medical packaging, in particular designed as a film bag or bottle, comprising a connector according to claim 1.

10. The medical packaging according to claim 9, characterized in that the lower part of the connector comprises a weld-in portion designed in particular ship-shaped, which is welded with the welding seam of a film bag in a fluid-tight manner.

11. A connection system, comprising a connector according claim 1 as well as a connection, which has a connection inner sleeve designed to engage into the inner sleeve of the connector, and which has a connection outer sleeve with an external thread.

12. The connection system according to claim 11, characterized in that the connection system is designed in such manner that the connection inner sleeve pushes the sealing head back when the connection is screwed into the connector, wherein the trunk portion expands in a hollow space formed by the gap and wherein the diameter of the sealing head enlarges such that the slit is opened and the channel is opened for the passage of the fluid.

13. The connection system according to claim 11, characterized in that the connection system is designed in such manner that, when the connection is screwed in, the front portion of the sealing head is pushed back into a region of the attachment part, in which the inner diameter of the attachment part, in particular behind a step, is enlarged.

14. The connection system according to claim 11, characterized in that the connection system is designed in such manner that the cross-section of the channel formed by the slit corresponds at least to the inner cross-section of the connection inner sleeve and/or in that the connection system is designed in such manner that the sealing element is resealed by returning to its original shape when the connection is unscrewed.

15. The connection system according claim 11, characterized in that the connection inner sleeve is designed conically and/or in that the slit has a length which is smaller than the outer diameter of the connection inner sleeve.

16. An apheresis set, comprising a connection system according to claim 11.

17. A method for providing a fluid connection to a medical packaging, in particular by means of a connection system according to claim 11, comprising the steps:
providing a connector, which comprises an attachment part with a sealing element, wherein the sealing element has a front surface with at least one slit, and wherein the attachment part has an outer sleeve which surrounds an inner sleeve and which has an internal thread,
providing a connection, which has a connection inner sleeve designed to engage into the inner sleeve of the connector, and a connection outer sleeve with an external thread,
screwing the connection into the connector, wherein the connection inner sleeve pushes the front surface of the sealing element back, wherein the sealing element, in particular a sealing head of the sealing element, is expanded and enlarges in diameter such that the slit opens into a channel, wherein an interior of the sealing element is free from of a body with a tip, which is intended to assist the opening of the sealing element.

18. The method according to claim 17, characterized in that the sealing element has a sealing head with a front portion, which comprises the front surface with the slit, wherein the front portion is adjoined by a rear portion, which has a larger diameter than the front portion, wherein the front portion is enlarged in its diameter behind a step of the attachment part when the sealing head is pushed back.

19. The method according to claim 17, characterized in that the connection is unscrewed again, wherein the sealing element returns to its original shape, wherein the fluid connection is closed by the diameter of the sealing element, in particular of a sealing head, being reduced, while a front surface of the connection inner sleeve still sits on the sealing element in a sealing manner.
